(19) United States
(12) Patent Application Publication
(10) Pub. No.: US 2019/0027256 A1
(43) Pub. Date: Jan. 24, 2019

(54) REMOTE HEALTHCARE SYSTEMS AND METHODS

(71) Applicant: AKOS MD, LLC, Phoenix, AZ (US)

(72) Inventors: Swaraj Singh, Glendale, AZ (US); Kishlay Anand, Paradise Valley, AZ (US)

(73) Assignee: AKOS MD, LLC, Phoenix, AZ (US)

(21) Appl. No.: 16/044,336

(22) Filed: Jul. 24, 2018

Related U.S. Application Data
(60) Provisional application No. 62/536,453, filed on Jul. 24, 2017.

(51) Int. Cl. G16H 80/00; G16H 10/60; G16H 10/20

(52) U.S. Cl. CPC G16H 80/00; G06F 3/0484; G16H 10/20; G16H 10/60

(57) ABSTRACT

A telemedicine system comprises a non-transitory, tangible computer-readable storage medium having instructions stored thereon that, in response to execution by a computer-based system, cause the computer-based system to perform operations comprising retrieving a work injury form from a healthcare database, selecting a first healthcare provider, contacting a first communication device operated by the first healthcare provider, creating a real-time communication link between the first communication device and a second communication device operated by an employee, providing graphical user interfaces the healthcare provider and the employee, receiving medical information of the employee, populating the work injury form with the medical information, and providing the work injury form to at least one of the first healthcare provider, an employer, or a regulatory body.

### CROSS REFERENCE TO RELATED APPLICATION

This non-provisional patent application claims priority to U.S. Provisional Patent Application No. 62/536,453, filed on Jul. 24, 2017, entitled “REMOTE HEALTHCARE SYSTEMS AND METHODS,” which is incorporated herein by reference in its entirety.

### FIELD

The present disclosure generally relates to remote healthcare systems and methods and, more particularly, to systems and methods for providing and receiving treatment and related healthcare for work-related injuries.

### BACKGROUND

Workers' compensation insurance provides certain medical and financial benefits to employees who are injured in the course of their employment, in exchange for a release of the employer's liability for certain claims. Workers' compensation systems are highly regulated, and the requirements for coverage and reporting vary by jurisdiction, by industry, and by the size and structure of the employer's business. Typically, when a covered employee is injured, he or she is evaluated and treated by a health care provider. Often, if medications are prescribed by a physician, the employee's injury must be reported to various regulatory bodies, and the employee's return to work may be delayed.

Telemedicine offers patients and physicians the convenience of real-time interaction, without the need to schedule an advanced appointment or travel to a doctor's office. With telemedicine, patients have improved access to healthcare providers in urgent medical situations or when their ability to travel is limited.

However, typical telemedicine platforms directly connect a patient to a physician, without first conducting triage of the patient's medical issues. Moreover typical telemedicine platforms do not have the functionality or resources to handle patients with work-related injuries. Accordingly, new systems and methods are needed for providing and receiving healthcare for work-related injuries.

### SUMMARY

The present invention provides a mobile application, article of manufacture, and other computer-based systems for providing healthcare to employees injured at or as a result of work. In embodiments, an article of manufacture comprises a non-transitory, tangible computer-readable storage medium having instructions stored thereon that, in response to execution by a computer-based system, cause the computer-based system to perform operations comprising retrieving, by the computer-based system, a work injury form from a healthcare database, creating, by the computer-based system, a real-time communication link between a first communication device operated by an employee and a second communication device operated by a first healthcare provider, providing, by the computer-based system, an employee graphical user interface comprising an employee input field and an employee audiovisual display field, wherein the first communication device displays the employee graphical user interface, providing, by the computer-based system, a healthcare provider graphical user interface comprising a healthcare provider input field and a healthcare provider audiovisual display field, wherein the second communication device displays the healthcare provider graphical user interface, receiving, by the computer-based system, medical information of the employee from at least one of the first communication device or the second communication device, populating, by the computer-based system, the work injury form with the medical information, and providing, by the computer-based system, the work injury form to at least one of the first healthcare provider, an employer, or a regulatory body.

In various embodiments, the article of manufacture further comprises authorizing, by the computer-based system, the employee to access the computer-based system. In various embodiments, the article of manufacture further comprises selecting, by the computer-based system, the first healthcare provider from a group of available healthcare providers, and contacting, by the computer-based system, the first healthcare provider. In various embodiments, the medical information comprises at least one of medical history information and injury information. In various embodiments, the first healthcare provider is not authorized to prescribe medication. In various embodiments, the article of manufacture further comprises receiving, by the computer-based system, medical treatment instructions from the first healthcare provider, and providing, by the computer-based system, the medical treatment instructions to the employee. In various embodiments, the article of manufacture further comprises selecting, by the computer-based system, a second healthcare provider from a group of available healthcare providers, contacting, by the computer-based system, the second healthcare provider, and creating, by the computer-based system, a real-time communication link between the first communication device and the third communication device.

In embodiments, a computer-based telemedicine system for work-related injuries comprises a processor, a tangible, non-transitory memory configured to communicate with the processor, the tangible, non-transitory memory having instructions stored thereon that, in response to execution by the processor, cause the processor to perform operations comprising retrieving, by the processor, a work injury form from a healthcare database, creating, by the processor, a real-time communication link between a first communication device operated by an employee and a second communication device operated by a first healthcare provider, providing, by the computer-based system, an employee graphical user interface comprising an employee input field and an employee audiovisual display field, wherein the first communication device displays the employee graphical user interface, providing, by the computer-based system, a healthcare provider graphical user interface comprising a healthcare provider input field and a healthcare provider audiovisual display field, wherein the second communication device displays the healthcare provider graphical user interface, receiving, by the processor, medical information of the employee from at least one of the first communication device or the second communication device, populating, by the processor, the work injury form with the medical information, and providing, by the processor, the work injury form to at least one of the first healthcare provider, an employer, or a regulatory body.

In various embodiments, the computer-based telemedicine system further comprises authorizing, by the processor, the employee to access the computer-based telemedicine system. In various embodiments, the computer-based telemedicine system further comprises selecting, by the processor, the first healthcare provider from a group of available healthcare providers, and contacting, by the processor, the first healthcare provider. In various embodiments, the medical information comprises at least one of medical history information and injury information. In various embodiments, the first healthcare provider is not authorized to prescribe medications. In various embodiments, the computer-based telemedicine system further comprises receiving, by the processor, medical treatment instructions from the first healthcare provider, and providing, by the processor, the medical treatment instructions to the employee. In various embodiments, the computer-based telemedicine system further comprises selecting, by the processor, a second healthcare provider from a group of available healthcare providers, contacting, by the processor, the second healthcare provider, and creating, by the processor, a real-time communication link between the first communication device and the third communication device.

In embodiments, a method comprises retrieving, by a computer-based system, a work injury form from a healthcare database, selecting, by the computer-based system, a first healthcare provider from a group of available healthcare providers, contacting, by the computer-based system, a first communication device operated by a first healthcare provider, creating, by the computer-based system, a real-time communication link between the first communication device and a second communication device operated by an employee, providing, by the computer-based system, an employee graphical user interface comprising an employee input field and an employee audiovisual display field, wherein the first communication device displays the employee graphical user interface, providing, by the computer-based system, a healthcare provider graphical user interface comprising a healthcare provider input field and a healthcare provider audiovisual display field, wherein the second communication device displays the healthcare provider graphical user interface, receiving, by the computer-based system, medical information of the employee from at least one of the first communication device or the second communication device, populating, by the computer-based system, the work injury form with the medical information, and providing, by the computer-based system, the work injury form to at least one of the first healthcare provider, an employer, or a regulatory body.

In various embodiments, the method further comprises authorizing, by the computer-based system, the employee to access the computer-based system. In various embodiments, the medical information comprises at least one of medical history information and injury information. In various embodiments, the first healthcare provider is not authorized to prescribe medications. In various embodiments, the method further comprises receiving, by the computer-based system, medical treatment instructions from the first healthcare provider, and providing, by the computer-based system, the medical treatment instructions to the employee. In various embodiments, the method further comprises selecting, by the computer-based system, a second healthcare provider from a group of available healthcare providers, contacting, by the computer-based system, the second healthcare provider, and facilitating, by the computer-based system, real-time communication between the employee and the second healthcare provider.

The contents of this summary section are provided only as a simplified introduction to the disclosure, and are not intended to be used to limit the scope of the appended claims.

### BRIEF DESCRIPTION OF THE DRAWINGS

The accompanying drawings are included to provide a further understanding of the present disclosure and are incorporated in, and constitute a part of, this specification, illustrate various embodiments, and together with the description, serve to explain the principles of the disclosure.

FIG. 1 illustrates a process of using a telemedicine system in accordance with various embodiments;

FIG. 2 illustrates an exemplary graphical user interface on a user device in accordance with various embodiments;

FIG. 3 illustrates another exemplary graphical user interface on a user device in accordance with various embodiments; and

FIG. 4 illustrates yet another exemplary graphical user interface on a user device in accordance with various embodiments.

### DETAILED DESCRIPTION

The following description is of various embodiments only, and is not intended to limit the scope, applicability or configuration of the present disclosure in any way. Rather, the following description is intended to provide a convenient illustration for implementing various embodiments including the best mode. As will become apparent, various changes may be made in the function and arrangement of the elements described in these embodiments without departing from the scope of the present disclosure or appended claims.

As used herein, the term “telemedicine” refers to the provision and receipt of medical diagnosis, treatment, consultation, and advice, as well as other forms of healthcare, via remote, electronic communication means. Telemedicine may allow a patient to access and communicate with a healthcare provider via phone, computer, or other audiovisual communication device, and may utilize one or more audio or video relay devices, such as a camera, so that the healthcare provider can visually and aurally observe the patient.

As used herein, “work-related injuries” refers to injuries sustained by employees who are working in the course of their employment. Triage, treatment, and/or reporting of work-related injuries may be regulated by workers' compensation insurance rules. Compliance with these regulations may require completion and submission of state- or industry-specific forms, medical diagnosis, treatment, and/or incident reporting to state or federal regulatory bodies.

In accordance with various embodiments, a computer-based telemedicine system for work-related injuries facilitates communication between an injured employee, his or her employer, one or more healthcare providers, and/or a regulatory body. In various embodiments, the computer-based telemedicine system comprises an aftermarket software program. In various embodiments, the computer-based telemedicine system comprises software as a service. However, the computer-based telemedicine system may comprise software embedded into a chip, may comprise firmware, and/or may be installed on a communication device in any suitable manner. The computer-based telemedicine system may further comprise one or more user devices comprising a graphical user interface (“GUI”). The computer-based telemedicine system may further comprise a processor in communication with the one or more user devices.

In various embodiments, the computer-based telemedicine system is installed on a first communication device and/or a second communication device. The computer-based telemedicine system may facilitate communication between the first communication device and the second communication device by electronic communication or data transmission means. The first communication device and/or the second communication device may have access to an Internet connection. The first communication device may transmit data to the second communication device via an Internet connection, over a local network, and/or through any other suitable means of transmitting data. A network may include any cloud, cloud computing system or electronic communications system or method which incorporates hardware and/or software components.

In various embodiments, the first communication device and/or the second communication device may comprise a smartphone, a personal computer, an electronic kiosk, and/or a tablet device. However, the first communication device and the second communication device may comprise any device capable of transmitting and/or receiving data.

In various embodiments, the computer-based telemedicine system further comprises a host server or other computing systems including a processor for processing digital data. The host server may be in communication with the first communication device and/or the second communication device. The host server may be in communication with various databases including, without limitation, patient data, healthcare provider data, regulatory body data, and/or like data useful in the operation of the system. As those skilled in the art will appreciate, user computer may include an operating system (e.g., Windows NT, Windows 95/98/2000, Windows XP, Windows Vista, Windows 7, OS2, UNIX, Linux, Solaris, MacOS, etc.) as well as various conventional support software and drivers typically associated with computers.

In various embodiments, the computer-based telemedicine system comprises a tangible, non-transitory memory configured to communicate with a processor. The memory may have instructions stored thereon that, in response to execution by the processor, cause the computer-based telemedicine system to perform various operations.

These operations performed by the computer-based telemedicine system improve the functioning of the computer. For example, by the user simply selecting his or her employer or state of employment, as opposed to manually inputting such data, the user performs fewer computer functions and provides less manual input, which saves on data storage and memory, therefore, speeding processing. Development and storage of state-, industry-, and/or employer-specific forms that can be filled out electronically (for example, through fillable .pdf technology), improves data storage requirements, memory requirements, and processing speeds. Similarly, automatic selection and/or retrieval of the relevant, state-, industry-, and/or employer-specific form in response to employee identity or data input similarly improves data storage requirements, memory requirements, and processing speeds. Additionally, by transmitting, storing, and accessing data using the processes described herein, the security of the data is improved, which decreases the risk of the computer or network from being compromised.

In various embodiments, the system and method may include a graphical user interface for dynamically relocating/rescaling obscured textual information or video of an underlying window to become automatically viewable to the user. By permitting textual information or video to be dynamically relocated based on an overlap condition, the computer's ability to display information is improved. More specifically, the method for dynamically relocating textual information within an underlying window displayed in a graphical user interface may comprise displaying a first window containing textual information or video in a first format within a graphical user interface on a computer screen; displaying a second window containing textual information or video within the graphical user interface; constantly monitoring the boundaries of the first window and the second window to detect an overlap condition where the second window overlaps the first window such that the textual information or video in the first window is obscured from a user's view; determining the textual information or video would not be completely viewable if relocated to an unobstructed portion of the first window; calculating a first measure of the area of the first window and a second measure of the area of the unobstructed portion of the first window; calculating a scaling factor which is proportional to the difference between the first measure and the second measure; scaling the textual information or video based upon the scaling factor; automatically relocating the scaled textual information or video, by a processor, to the unobscured portion of the first window in a second format during an overlap condition so that the entire scaled textual information or video is viewable on the computer screen by the user; and automatically returning the relocated scaled textual information or video, by the processor, to the first format within the first window when the overlap condition no longer exists.

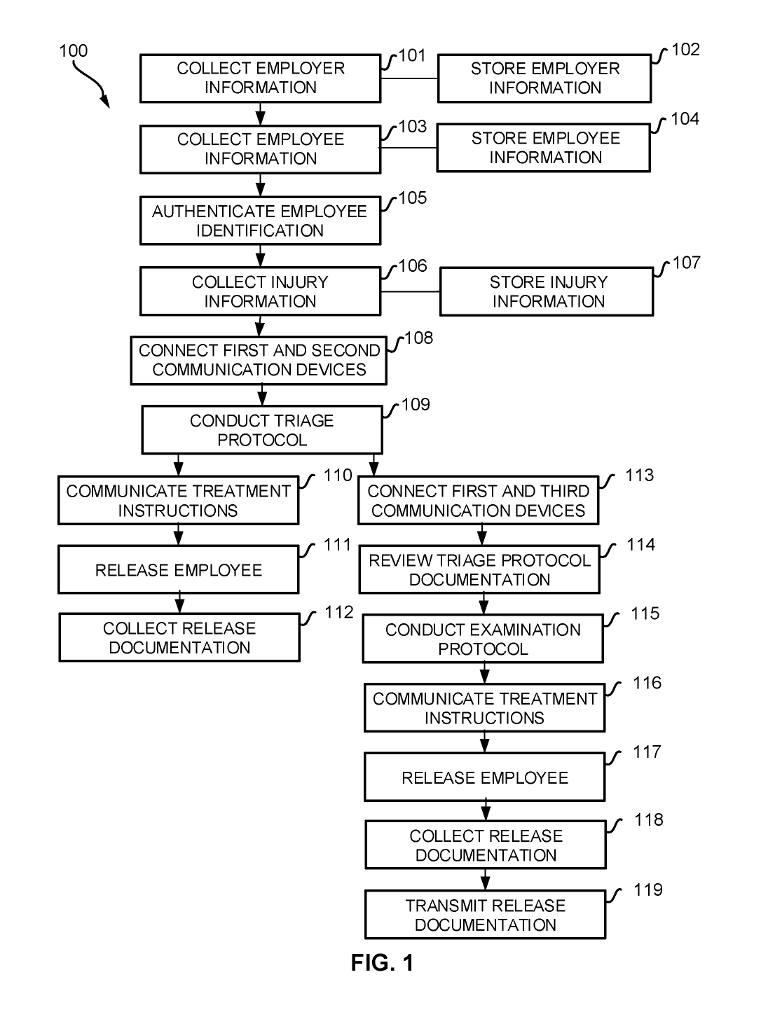

Referring to FIG. 1, a process of using a computer-based telemedicine system (100) is illustrated. In various embodiments, the computer-based telemedicine system collects (Step 101) and stores (Step 102) employer identifying information. Such information may be stored on any suitable server and/or network. Employer identifying information may comprise the employer's state of incorporation, residence, and/or operation. Employer identifying information may comprise information regarding the employer's industry, number of employees, governing regulations, and/or history of work-related injuries. Employer identifying information may comprise the names and/or other identifying information of one or more employees. In various embodiments, the computer-based telemedicine system may assign an identifying code to an employer and/or employee.

In various embodiments, the computer-based telemedicine system collects (Step 103) and stores (Step 104) employee identifying information. Employee identifying information may comprise the employee's name, age, address, signature, financial information, job title, job responsibilities, medical history, history of work-related injuries, and/or any other employment-related or medical information suitable for collection and storage by the computer-based telemedicine system.

In various embodiments, the computer-based telemedicine system authenticates an employee's identity (Step 105). In various embodiments, the authenticating may comprise comparing, by the processor, a first identifying code assigned to an employer and/or employee to a second identifying code input by the employer on a first communication device. In response to the first identifying code matching the second identifying code, the computer-based telemedicine system may retrieve, access, and/or send employee identifying information to the first communication device and/or the second communication device. In various embodiments, in response to the first identifying code matching the second identifying code, the computer-based telemedicine system identifies forms required by applicable regulations to document and treat a work-related injury. The forms may comprise state-, industry-, and/or employer-specific forms. In various embodiments, the computer-based telemedicine system transmits the forms to the first communication device and/or the second communication device, to be viewed by the employee, the employer, a healthcare provider, and/or a regulatory body.

In various embodiments, the computer-based telemedicine system encrypts data stored on and/or transmitted between the first communication device and/or the second communication device. Data may be encrypted using known encryption methods, such as Advanced Encryption Standard (“AES”) or RSA encryption. In various embodiments, AES 256-bit encryption is used. However, data may be encrypted using full-disk encryption, virtual disk encryption, file encryption, SSL encryption, or any other form of encryption suitable for use in a computer-based telemedicine system. In various embodiments, the computer-based telemedicine system may collect, store, and transmit data in compliance with HIPAA and any other applicable regulations.

In various embodiments, the computer-based telemedicine system collects from the employee (Step 106) and stores by the processor (Step 107) injury information. Such information may be stored on any suitable server and/or network. Injury information may comprise the time, date, nature, and/or extent of employee's injury. Injury information may comprise the employee's medical history, complications, and/or symptoms of the employee's injury. However, injury information may comprise any information helpful for the diagnosis and/or treatment of the injury and/or any information necessary for compliance with applicable regulations.

In various embodiments, the computer-based telemedicine system brings the first communication device into communication with the second communication device (Step 108) by any suitable means. In various embodiments, the computer-based telemedicine system automatically connects the first communication device to the second communication device in response to a command from the employee. In various embodiments, one or more healthcare providers may be available, or “on call,” to communicate with the employee on a second and/or third communication device. In various embodiments, the presence of “on call” healthcare providers minimizes or eliminates the time that an employee must wait for evaluation, diagnosis, and/or treatment by a healthcare provider.

In various embodiments, the first communication device may share an online folder with the second communication device. The computer-based telemedicine system may grant first communication device and/or second communication device access to the online folder. In various embodiments, access may be granted in response to exchange of authentication tokens, keys, web tokens, and/or any other suitable form of authentication. Data may be uploaded from first communication device and/or second communication device to the online folder, and/or data may be downloaded to the first communication device and/or the second communication device from the online folder.

In various embodiments, communication between the first communication device and the second communication device comprises an audio and/or visual connection. The employee may use the first communication device to see and speak with a first healthcare provider. The first healthcare provider may use the second communication device to see and speak with the employee.

In various embodiments, the first healthcare provider comprises a nurse and/or other healthcare provider who is not authorized to prescribe controlled medications. However, the first healthcare provider may comprise any suitable healthcare provider. As used herein, the term “controlled medications” refers to pharmaceuticals that may not be purchased by the public without a prescription or other form of healthcare provider authorization. The first healthcare provider may conduct a triage protocol via the computer-based telemedicine system (Step 109). The triage protocol may comprise an interview and/or examination of the employee by the first healthcare provider. The triage protocol may comprise collection and storage of employee medical information by the computer-based telemedicine system.

In response to the triage protocol, the first healthcare provider may communicate injury treatment instructions to the employee via the computer-based telemedicine system (Step 110). In various embodiments, the treatment instructions may be stored by the computer-based telemedicine system. Such information may be stored on any suitable server and/or network. In response to treatment instructions that do not include the prescription of controlled medications, the first healthcare provider may instruct the computer-based telemedicine system to release the employee (Step 111) and/or may complete release documentation (Step 112). Release documentation may comprise forms required by the employer and/or applicable regulations to be completed, stored, and/or provided to regulatory bodies in the event of a work-related injury. Such information may be stored on any suitable server and/or network.

In response to the triage protocol, the computer-based telemedicine system may place the first communication device into communication with a third communication device (Step 113). In various embodiments, the computer-based telemedicine system may place the first communication device into communication with a third communication device in response to a command from the first healthcare provider. In various embodiments, the computer-based telemedicine system may place the first communication device into communication with a third communication device in response to the healthcare provider indicating that a controlled prescription is necessary. In various embodiments, the computer-based telemedicine system may place the first communication device into communication with a third communication device in response to the healthcare provider requiring assistance in diagnosis and/or treatment of the employee.

The third communication device may comprise a device with functionality and features similar to the first communication device and the second communication device. In various embodiments, communication between the first communication device and the third communication device comprises an audio and/or visual connection. The employee may use the first communication device to see and speak with a second healthcare provider. The second healthcare provider may use the third communication device to see and speak with the employee.

In various embodiments, the second healthcare provider comprises a physician, physician assistant, nurse practitioner, and/or any other healthcare provider who is authorized to prescribe controlled medications. However, the second healthcare provider may comprise any suitable healthcare provider. The second healthcare provider may use the computer-based telemedicine system to access and review triage protocol information collected by the first healthcare provider (Step 114). The second healthcare provider may use the computer-based telemedicine system to conduct an examination protocol (Step 115) comprising interview and/or examination of the employee by the second healthcare provider.

In response to the examination protocol, the second healthcare provider may communicate injury treatment instructions to the employee via the computer-based telemedicine system (Step 116). In various embodiments, the treatment instructions may be stored by the computer-based telemedicine system. Such information may be stored on any suitable server and/or network. The second healthcare provider may instruct the computer-based telemedicine system to release the employee (Step 117) and/or may complete release documentation (Step 118). Release documentation may comprise forms required by the employer and/or applicable regulations to be completed, stored, and/or provided to regulatory bodies in the event of a work-related injury. Such information may be stored on any suitable server and/or network.

In response to treatment instructions that include the prescription of a controlled medication, the computer-based telemedicine system may transmit release documentation to the employer and/or an applicable regulatory body (Step 119). In various embodiments, treatment instructions that include the prescription of a controlled medication may constitute a reportable incident that is required to be reported to a regulatory body. In various embodiments, treatment instructions that do not include the prescription of a controlled medication may not constitute a reportable incident.

In various embodiments, triage and treatment of the employee by the first healthcare provider via the computer-based telemedicine increases the likelihood that the employee will be released without prescription of a controlled medication, as compared to assessment and treatment of injured employees in a traditional healthcare setting, such as a doctor's office, emergency room, urgent care facility, or the like.

In various embodiments, the computer-based telemedicine system may comprise a GUI viewable by users. The GUI may comprise a content navigation system which provides an intuitive and uncluttered user experience. The GUI may allow a user, even with limited expertise, to navigate the computer-based telemedicine system using a hypertext-based method, a lexical (dictation, typing) data entry method, or any other suitable method. A user may select hypertext-based data elements by any known method, such as by tapping on a touch-screen, swiping, clicking with a pointer, speaking a command, etc. The GUI may parse complex medical scenarios into a manageable and non-distracting format optimized for use on a mobile device, a touch screen interface, a laptop computer, and/or a desktop computer.

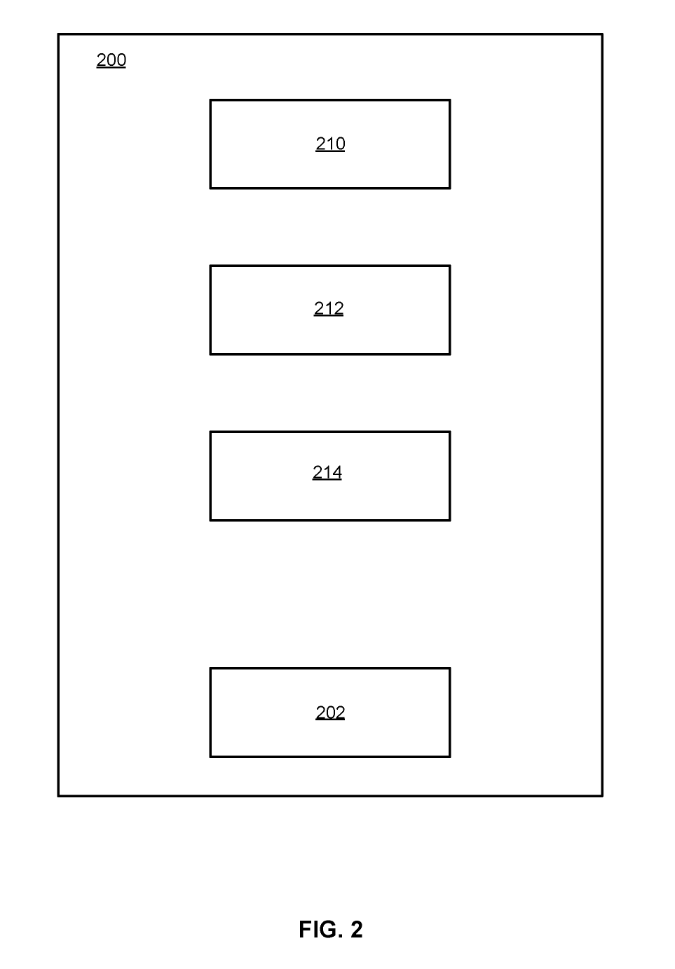

With reference now to FIG. 2, GUI 200 may comprise one or more means by which users may input data, documents, medical information, identifying information or other information or materials into the computer-based telemedicine system. In various embodiments, GUI 200 comprises one or more text entry fields 210. Text entry field 210 may be fillable and/or editable by lexical data entry, by drop-down menu selection, or by any other suitable method. GUI 200 may display instructional prompts in or near text entry field 210 to guide a user's data entry. In various embodiments, GUI 200 comprises one or more data selection hypertexts 212. A user may click, push, command or otherwise select data selection hypertext 212 in order to provide information to the computer-based telemedicine system.

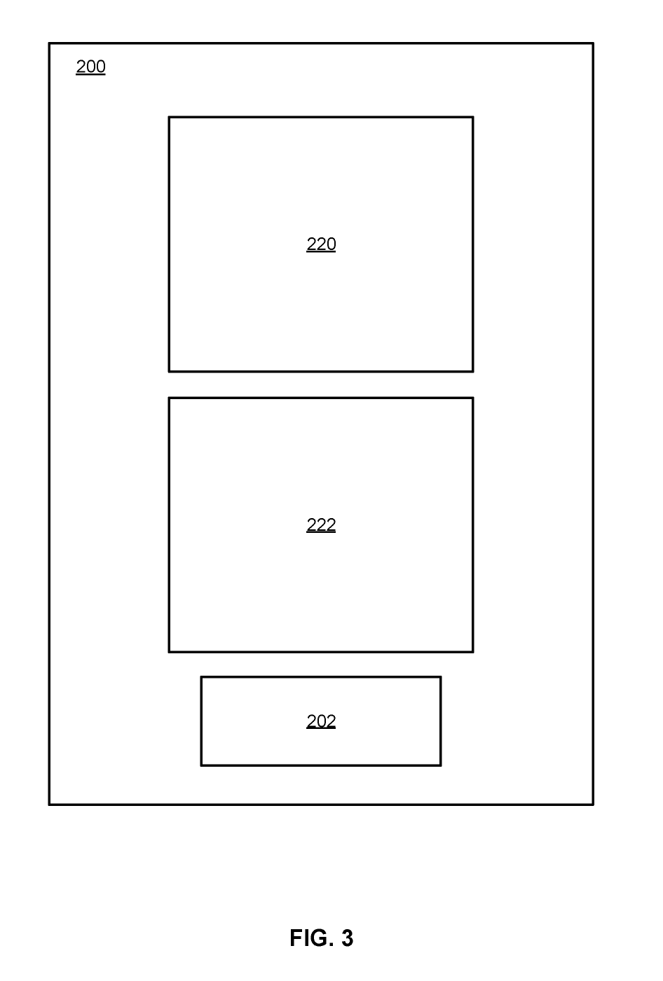

In various embodiments, GUI 200 comprises a document upload field 214. Document upload field 214 may enable a user to input an image, such as a photograph or drawing. The user may select document upload field 214, and the user may take a photograph, use a stored photograph, or create a drawing in freehand entry field 222, with momentary reference to FIG. 3. Thus, the user may be able to quickly photograph an employee's condition and store and/or upload the photograph to the computer-based telemedicine system. Metadata associated with the image may indicate that the image is associated with the incident and/or an injury alleged to have occurred in connection therewith. For example, the system may concatenate metadata with the image data indicating that the image is associated with swelling or bruising. Document upload field 214 may enable a user to input medical records, injury documentation, bills, insurance information, and/or any other documents usable by the computer-based telemedicine system.

GUI 200 may display text entry field 210, data selection hypertext 212, or document upload field 214 to facilitate gathering of information related to: (1) an employee's identifying information, authentication information, employer information, work history, incident information, injury information, preferred pharmacy information, medical history, family history, and social history; (2) an employer's employee information, reportable incident history, job descriptions, and incident reporting requirement; (3) a healthcare provider's contact information, schedule availability, credentials, treatment instructions, exam results, medical diagnosis, and miscellaneous patient observations; and/or (4) any other information usable by the computer-based telemedicine system.

In various embodiments, GUI 200 comprises one or more navigational hypertexts 202 configured to allow a user to advance to subsequent pages or interfaces of GUI 200, or to return to previously viewed pages or interfaces of GUI 200.

In various embodiments and with reference now to FIG. 3, GUI 200 comprises an audiovisual display field 220. Audiovisual display field 220 may be configured to allow real-time audiovisual communication between remotely-located users of the computer-based telemedicine system including, without limitation, one or more employees, employers, and healthcare providers. Audiovisual display field 220 may facilitate one-way, two-way, or multi-way visualization of computer-based telemedicine system users. Audiovisual display field 220 may display to a first user both real-time footage of a remotely-located second user and real-time footage of the first user.

In various embodiments, GUI 200 comprises one or more freehand entry fields 222. Freehand entry field 222 may be configured to record a user-generated two-dimensional figure, such as a signature, diagram, and/or drawing. Freehand entry field 222 may be configured to capture a drawing or depiction of an employee injury, workplace floor plan, or other incident or injury information provided by an employer, employee, and/or witness to the incident. Freehand entry field 222 may be configured to capture a signature of an employer, employee, witness, healthcare provider, and/or any other computer-based telemedicine system user.

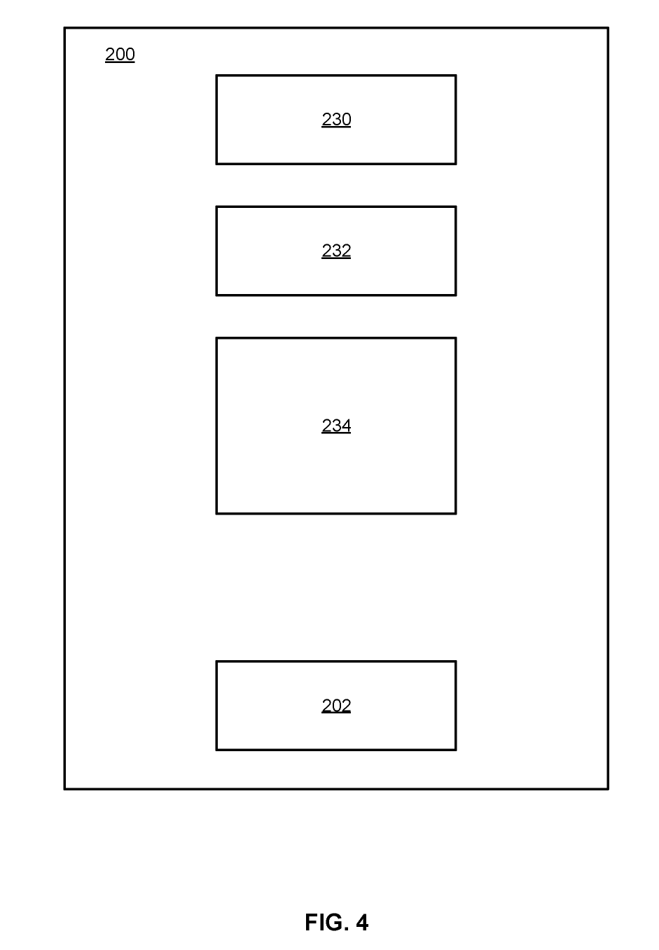

With reference now to FIG. 4, in various embodiments, GUI 200 comprises one or more tabs 230, links 232, and/or chat windows 234. Tab 230 may comprise a selectable hyperlink configured to display a page, field, or other discreet portion of GUI 200 to facilitate a user's navigation of the computer-based telemedicine system. Link 232 may comprise a hyperlink to an external webpage, document, or other electronic resource. Link 232 may comprise a hyperlink to another portion or page of the computer-based telemedicine system itself. Chat window 234 may be configured to facilitate real-time textual communication between a first user of the computer-based telemedicine system, and a second user or administrator of the computer-based telemedicine system.

In various embodiments, the computer-based telemedicine system comprises a login page. The login page may be viewable to employers, employees, and/or healthcare providers. The login page may request an email address, user identification credential, password, and/or any other identification or credential suitable to identify a user of the computer-based telemedicine system. The login page may include a link by which new users of the computer-based telemedicine system may sign up for services and/or login credentials.

In various embodiments, the computer-based telemedicine system may comprise one or more prompt pages. A first prompt page may display a question to a user and/or buttons by which the user may answer the displayed question. In various embodiments, the first prompt page may ask a user whether the computer-based telemedicine system services are provided to the user by the user's employer.

In various embodiments, the computer-based telemedicine system may comprise one or more authentication pages. A first authentication page may be displayed to users in response to a signal that an employee receives computer-based telemedicine system services through his or her employer. The first authentication page may request that the user enter an employer identification code and/or an employee identification code. The employer identification code and/or an employee identification code may have been previously provided to the employee by his or her employer or by an administrator of the computer-based telemedicine system. In various embodiments, the computer-based telemedicine system may retrieve profile information in response to entry of the employer identification code and/or the employee identification code may. The profile information may include the employer's name, address, state of incorporation, state of operation, and/or industry, the employee's name, address, medical history, work-related injury history, job title, and/or job requirements, and/or any other relevant information previously received and/or stored by the computer-based telemedicine system.

In various embodiments, the computer-based telemedicine system may comprise a profile page. The profile page may display windows or boxes suitable for receiving and/or displaying employee information and/or employer information including, without limitation, a user's login, name, email address, phone number, physical address, work address, gender, marital status, and/or any other information suitable for use in a computer-based telemedicine system. The profile page may display information previously received and/or stored by the computer-based telemedicine system. The profile page may allow a user to input new information, delete displayed information, and/or edit displayed information.

In various embodiments, the computer-based telemedicine system comprises a password page. The password page may allow a user to input and confirm a password, PIN, and/or any other personal identifier.

In various embodiments, the computer-based telemedicine system may comprise a verification page. The verification page may display information to a user confirming that the user has successfully created a profile and/or requesting verification that the user's selected email address is authentic.

In various embodiments, the computer-based telemedicine system may comprise a second prompt page. The second prompt page may display a question to a user and/or buttons by which the user may answer the displayed question. In various embodiments, the second prompt page may ask a user to indicate what type of medical care the user is requesting. The second prompt page may allow a user to indicate that he or she is requesting medical care, pediatric care, and/or care related to a workplace injury.

In various embodiments, the computer-based telemedicine system comprises an injury information page. The injury information page may display windows or boxes in which an employee can input personal information and/or information regarding a work-related injury. The computer-based telemedicine system may receive and/or store photographs related to the employee's injury. The computer-based telemedicine system may receive and/or store additional injury information including, without limitation, the time, location, details, and/or witnesses of the work-related injury, the employee's social security number or other personal identifying information, and/or the name and contact information of the employee's supervisor.

In various embodiments, the computer-based telemedicine system may comprise a signature page. The signature page may receive a user's signature. In various embodiments, the user may draw his or her signature on a surface of a device having touch screen capabilities. In various embodiments, the user may upload or otherwise transmit to the computer-based telemedicine system a file containing or displaying the user's signature.

In various embodiments, the computer-based telemedicine system comprises one or more medical history pages. The medical history page may receive a user's medical history information including, without limitation, a user's current medications, drug allergies, preexisting medical conditions, surgical history, work-related injury history, and/or any other information relevant to a user's medical history.

In various embodiments, the computer-based telemedicine system comprises a third prompt page. The third prompt page may display a question to a user and/or buttons by which the user may answer the displayed question. In various embodiments, the third prompt page may ask a user to indicate how an employee wishes to communication with a healthcare provider. The third prompt page may allow a user to indicate that he or she is requesting an audio-only connection to a healthcare provider and/or an audiovisual connection to a healthcare provider.

In various embodiments, the computer-based telemedicine system may store information input by a user on a login page, prompt page, authentication page, profile page, password page, verification page, injury information page, signature page, and/or medical history page. The computer-based telemedicine system may display such information to an employee, employer, and/or healthcare provider. The computer-based telemedicine system may copy such information into one or more forms required by applicable regulations to document and treat a work-related injury.

In various embodiments, the computer-based telemedicine system comprises a first care request page. In response to a user requesting communication with a healthcare provider, the computer-based telemedicine system may display a first care request page to a second communication device and/or to a healthcare provider using a second communication device. The computer-based telemedicine system may maintain a connection with more than one communication device and/or may display the first care request page to more than one communication device. In various embodiments, the computer-based telemedicine system may display the first care request page to a limited group of healthcare providers such as, for example, nurses, medical assistants, and/or other healthcare providers not authorized to prescribe controlled medications. The first care request page may display injury information including, without limitation, the employee's name, gender, age, location, injury, symptoms, and/or medical history. The first care request page may display a question to the healthcare provider and/or buttons by which the healthcare provider may indicate whether he or she will accept or decline to communicate with the employee. In response to the healthcare provider accepting, the computer-based telemedicine system may place the employee's communication device in communication with the healthcare provider's communication device.

In various embodiments, the computer-based telemedicine system may display to a healthcare provider a real-time audiovisual connection to the employee, injury information and/or other information received by the computer-based telemedicine system from the employee, the employee's job description, and/or any other information regarding the employer or the employee. In various embodiments, the computer-based telemedicine system may comprise one or more windows, boxes, tabs, pages or the like into which the healthcare provider may input information regarding triage, his or her observations of the employee, the employee's injury, diagnosis, and/or treatment, the employee's preferred pharmacy, instructions provided to the employee, and/or any other information relevant to the work-related injury. In various embodiments, the computer-based telemedicine system may allow the healthcare provider to edit, delete, and/or update any information previously provided by the employee. In various embodiments, the computer-based telemedicine system may display to the healthcare provider a list of physicians to whom the employee may be referred for additional medical treatment.

In various embodiments, the computer-based telemedicine system may comprise a second care request page. In response to a first healthcare provider requesting referral to a second healthcare provider, the computer-based telemedicine system may display a second care request page to a third communication device and/or to a healthcare provider using a third communication device. The computer-based telemedicine system may maintain a connection with more than one communication device and/or may display the second care request page to more than one communication device. In various embodiments, the computer-based telemedicine system may display the second care request page to a limited group of healthcare providers such as, for example, physicians, nurse practitioners, and/or other healthcare providers authorized to prescribe controlled medications. The second care request page may display injury information including, without limitation, the employee's name, gender, age, location, injury, symptoms, and/or medical history. The second care request page may display a question to the second healthcare provider and/or buttons by which the second healthcare provider may indicate whether he or she will accept or decline to communicate with the employee. In response to the second healthcare provider accepting, the computer-based telemedicine system may place the employee's communication device in communication with the second healthcare provider's communication device.

In various embodiments, the computer-based telemedicine system may display to the second healthcare provider a real-time audiovisual connection to the employee, injury information and/or other information received by the computer-based telemedicine system from the employee and the first healthcare provider, the employee's job description, and/or any other information regarding the employer or the employee. In various embodiments, the computer-based telemedicine system may allow the second healthcare provider to edit, delete, and/or update any information previously provided by the employee and the first healthcare provider. In various embodiments, the computer-based telemedicine system may comprise one or more windows, boxes, tabs, pages or the like into which the second healthcare provider may input information regarding triage, his or her observations of the employee, the employee's injury, diagnosis, and/or treatment, the employee's preferred pharmacy, instructions provided to the employee, medications prescribed to the employee, and/or any other information relevant to the work-related injury.

In various embodiments, the computer-based telemedicine system may allow the employee and/or a healthcare provider to end the connection between the first communication device and the second communication device. In various embodiments, in response to disconnection, the computer-based telemedicine system may display a feedback prompt to the employee and/or to the healthcare provider. The feedback prompt may allow the user to indicate whether the communication was successful and/or to provide written feedback related to the user's experience.

In various embodiments, the computer-based telemedicine system may comprise various tools with which an employee and/or a healthcare provider may analyze, monitor, and document use of the computer-based telemedicine system. In various embodiments, the computer-based telemedicine system comprises features for allowing a user to convert collected and/or stored information into a printable electronic form. In various embodiments, the computer-based telemedicine system comprises features for allowing a user to transmit via email information collected and/or stored by the computer-based telemedicine system.

In various embodiments, the computer-based telemedicine system may comprise a dashboard. The dashboard may display a list of communications facilitated by the computer-based telemedicine system. The list may display information related to an employer, and employee, and/or a healthcare provider.

In various embodiments, the computer-based telemedicine system comprises a call log. The call log may allow a user to access, review, edit, and/or update information related to a single, identified communication facilitated by the telemedicine system.

In various embodiments, the computer-based telemedicine system comprises a claim form page. The claim form page may comprise windows and/or boxes into which a user may input information required by a state-, industry-, and/or employer-specific form required in response to a work-related injury. The computer-based telemedicine system may display information previously collected and/or stored by the computer-based telemedicine system on the claim form page. In various embodiments, the computer-based telemedicine system may display a user's signature in connection with the claim form page.

In various embodiments, the computer-based telemedicine system comprises a converted claim form page. The converted claim form page may display information previously collected and/or stored on a state-, industry-, and/or employer-specific form required in the event of a work-related injury. The converted claim form page may comprise an electronic format suitable for printing, downloading, and/or transmission via email.

In various embodiments, the computer-based telemedicine system comprises features for transmitting forms, release documentation, and/or any other information collected by the computer-based telemedicine system. The computer-based telemedicine system may direct transmission to the employer, the employee, a healthcare provider, an insurer, a regulatory body, and/or any other suitable recipient. Transmission may occur via email, via access to the computer-based telemedicine system, via an online folder, or via any other suitable method of transmission. In various embodiments, the computer-based telemedicine system comprises features for communicating with a printer and/or instructing the printer to print forms, release documentation, and/or any other information collected by the computer-based telemedicine system.

The present system or any part(s) or function(s) thereof may be implemented using hardware, software or a combination thereof and may be implemented in one or more computer systems or other processing systems. However, the manipulations performed by embodiments were often referred to in terms, such as selecting and authorizing, which are commonly associated with mental operations performed by a human operator. No such capability of a human operator is necessary, or desirable in most cases, in any of the operations described herein. Rather, the operations may be machine operations. Useful machines for performing the various embodiments include general purpose digital computers or similar devices.

It should be appreciated that exemplary components and steps may be realized by any number of hardware, software, or other components configured to perform the specified functions. For example, an exemplary embodiment employs various graphical user interfaces, software components, and networking and/or database functionality. In addition, various embodiments may be practiced in any number of medical record management and/or information management contexts, and the embodiments disclosed are merely indicative of exemplary applications. For example, the principles, features and methods discussed may be applied to various industries, and are not limited to use in connection with health records and/or workers' compensation systems.

The detailed description of various embodiments herein makes reference to the accompanying drawings and pictures, which show various embodiments by way of illustration. While these various embodiments are described in sufficient detail to enable those skilled in the art to practice the disclosure, it should be understood that other embodiments may be realized and that logical and/or functional changes may be made without departing from the spirit and scope of the disclosure. Thus, the detailed description herein is presented for purposes of illustration only and not of limitation. For example, the steps recited in any of the method or process descriptions may be executed in various orders and are not limited to the order presented. Moreover, certain of the functions or steps may be outsourced to or performed by one or more third parties. Furthermore, any reference to singular includes plural embodiments, and any reference to more than one component may include a singular embodiment.

Systems, methods and computer program products are provided. In the detailed description herein, references to “an exemplary embodiment,” “various embodiments,” “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

In various embodiments, the methods described herein are implemented using the various particular machines described herein. The methods described herein may be implemented using the below particular machines, and those hereinafter developed, in any suitable combination, as would be appreciated immediately by one skilled in the art. Further, as is unambiguous from this disclosure, the methods described herein may result in various transformations of certain articles.

For the sake of brevity, conventional techniques for data networking, software application development, cloud computing, and/or the like, may not be described in detail herein. Furthermore, the connecting lines shown in various figures contained herein are intended to represent exemplary functional relationships and/or physical or communicative couplings between various elements. It should be noted that many alternative or additional functional relationships or physical or communicative connections may be present in a practical health records management system.

As used herein, “cloud” or “cloud computing” includes a model for enabling convenient, on-demand network access to a shared pool of configurable computing resources (e.g., networks, servers, storage, applications, and services) that can be rapidly provisioned and released with minimal management effort or service provider interaction. Cloud computing may include location-independent computing, whereby shared servers provide resources, software, and data to computers and other devices on demand.

As used herein, the term “network” includes any cloud, cloud computing system or electronic communications system or method which incorporates hardware and/or software components. Communication among the parties may be accomplished through any suitable communication channels, such as, for example, a telephone network, an extranet, an intranet, Internet, point of interaction device [point of sale device, personal digital assistant (e.g., iPhone®, Blackberry®), cellular phone, kiosk, etc.], online communications, satellite communications, off-line communications, wireless communications, transponder communications, local area network (LAN), wide area network (WAN), virtual private network (VPN), networked or linked devices, keyboard, mouse and/or any suitable communication or data input modality. Moreover, although the system is frequently described herein as being implemented with TCP/IP communications protocols, the system may also be implemented using IPX, AppleTalk®, IP-6, NetBIOS®, OSI, any tunneling protocol (e.g. IPsec, SSH), or any number of existing or future protocols. If the network is in the nature of a public network, such as the Internet, it may be advantageous to presume the network to be insecure and open to eavesdroppers. Specific information related to the protocols, standards, and application software utilized in connection with the Internet is generally known to those skilled in the art and, as such, need not be detailed herein. See, e.g., Dilip Naik, Internet Standards and Protocols (1998); JAVA® 2 Complete, various authors, (Sybex 1999); Deborah Ray and Eric Ray, Mastering HTML 4.0 (1997); Loshin, TCP/IP Clearly Explained (1997); and David Gourley and Brian Totty, HTTP, The Definitive Guide (2002), the contents of which are hereby incorporated by reference.

As used herein, “transmit” may include sending electronic data from one system component to another over a network connection. Additionally, as used herein, “data” may include encompassing information such as commands, queries, files, data for storage, and the like in digital or any other form.

Communication among the parties may be accomplished through any suitable communication channels, such as, for example, a telephone network, an extranet, an intranet, Internet, point of interaction device, point of sale device, personal digital assistant (e.g., iPhone®, PalmPilot®, Blackberry®, cellular phone, smart phone, kiosk, etc.), online communications, satellite communications, off-line communications, wireless communications, transponder communications, local area network (LAN), wide area network (WAN), virtual private network (VPN), networked or linked devices, keyboard, mouse and/or any suitable communication or data input modality. Moreover, although the system is frequently described herein as being implemented with TCP/IP communications protocols, the system may also be implemented using IPX, AppleTalk®, IP-6, NetBIOS, OSI, any tunneling protocol (e.g. IPsec, SSH), or any number of existing or future protocols. If the network is in the nature of a public network, such as the Internet, it may be advantageous to presume the network to be insecure and open to eavesdroppers. Specific information related to the protocols, standards, and application software utilized in connection with the Internet is generally known to those skilled in the art and, as such, need not be detailed herein. See, for example, DILIP NAIK, INTERNET STANDARDS AND PROTOCOLS (1998); JAVA 2 COMPLETE, various authors, (Sybex 1999); DEBORAH RAY AND ERIC RAY, MASTERING HTML 4.0 (1997); and LOSHIN, TCP/IP CLEARLY EXPLAINED (1997) and DAVID GOURLEY AND BRIAN TOTTY, HTTP, THE DEFINITIVE GUIDE (2002), the contents of which are hereby incorporated by reference.

The various system components may be independently, separately or collectively suitably coupled to the network via data links which includes, for example, a connection to an Internet Service Provider (ISP) over the local loop as is typically used in connection with standard modem communication, cable modem, dish networks, ISDN, Digital Subscriber Line (DSL), or various wireless communication methods, see, e.g., GILBERT HELD, UNDERSTANDING DATA COMMUNICATIONS (1996), which is hereby incorporated by reference. It is noted that the network may be implemented as other types of networks, such as an interactive television network.

In fact, in various embodiments, the embodiments are directed toward one or more computer systems capable of carrying out the functionality described herein. The computer-based telemedicine system may include one or more processors, such as a processor for generating medical informatics documentation. The processor is connected to a communication infrastructure (e.g., a communications bus, cross over bar, or network). Various software embodiments are described in terms of this exemplary computer-based telemedicine system. After reading this description, it will become apparent to a person skilled in the relevant art(s) how to implement various embodiments using other computer systems and/or architectures. The computer-based telemedicine system can include a display interface that forwards graphics, text, and other data from the communication infrastructure (or from a frame buffer not shown) for display on a display unit.

The computer-based telemedicine system may also include a main memory, such as for example random access memory (RAM), and may also include a secondary memory. The secondary memory may include, for example, a hard disk drive and/or a removable storage drive, representing a floppy disk drive, a magnetic tape drive, an optical disk drive, etc. The removable storage drive reads from and/or writes to a removable storage unit in a well-known manner. Removable storage unit represents a floppy disk, magnetic tape, optical disk, etc. which is read by and written to by removable storage drive. As will be appreciated, the removable storage unit includes a computer usable storage medium having stored therein computer software and/or data.

In various embodiments, software may be stored in a computer program product and loaded into computer system using removable storage drive, hard disk drive or communications interface. The control logic (software), when executed by the processor, causes the processor to perform the functions of various embodiments as described herein. In various embodiments, the computer-based telemedicine system comprises hardware components such as application specific integrated circuits (ASICs). Implementation of the hardware state machine so as to perform the functions described herein will be apparent to persons skilled in the relevant art(s).

In various embodiments, components, modules, and/or engines of system may be implemented as micro-applications or micro-apps. Micro-apps are typically deployed in the context of a mobile operating system, including for example, a Windows® mobile operating system, an Android® Operating System, Apple® iOS®, a Blackberry® operating system and the like. The micro-app may be configured to leverage the resources of the larger operating system and associated hardware via a set of predetermined rules which govern the operations of various operating systems and hardware resources. For example, where a micro-app desires to communicate with a device or network other than the mobile device or mobile operating system, the micro-app may leverage the communication protocol of the operating system and associated device hardware under the predetermined rules of the mobile operating system. Moreover, where the micro-app desires an input from a user, the micro-app may be configured to request a response from the operating system which monitors various hardware components and then communicates a detected input from the hardware to the micro-app.

As used herein, “computer-readable storage medium” does not include transitory phenomena such as propagating electromagnetic signals. The term “non-transitory” is to be understood to remove only propagating transitory signals per se from the claim scope and does not relinquish rights to all standard computer-readable media that are not only propagating transitory signals per se. Stated another way, the meaning of the term “non-transitory computer-readable medium” and “non-transitory computer-readable storage medium” should be construed to exclude only those types of transitory computer-readable media which were found in In Re Nuijten to fall outside the scope of patentable subject matter under 35 U.S.C. § 101.

While the steps outlined herein represent exemplary embodiments of principles of the present disclosure, practitioners will appreciate that there are any number of computing algorithms and user interfaces that may be applied to create similar results. The steps are presented for the sake of explanation only and are not intended to limit the scope of the present disclosure in any way. Benefits, other advantages, and solutions to problems have been described herein with regard to specific embodiments. However, the benefits, advantages, solutions to problems, and any element(s) that may cause any benefit, advantage, or solution to occur or become more pronounced are not to be construed as critical, required, or essential features or elements of any or all of the claims.

It should be understood that the detailed description and specific examples, indicating exemplary embodiments, are given for purposes of illustration only and not as limitations. Many changes and modifications may be made without departing from the spirit thereof, and principles of the present disclosure include all such modifications. Corresponding structures, materials, acts, and equivalents of all elements are intended to include any structure, material, or acts for performing the functions in combination with other elements. Reference to an element in the singular is not intended to mean “one and only one” unless explicitly so stated, but rather “one or more.” Moreover, when a phrase similar to “at least one of A, B, or C” or “at least one of A, B, and C” is used in the claims or the specification, the phrase is intended to mean any of the following: (1) at least one of A; (2) at least one of B; (3) at least one of C; (4) at least one of A and at least one of B; (5) at least one of B and at least one of C; (6) at least one of A and at least one of C; or (7) at least one of A, at least one of B, and at least one of C.

1. An article of manufacture, comprising:
a non-transitory, tangible computer-readable storage medium having instructions stored thereon that, in response to execution by a computer-based system, cause the computer-based system to perform operations comprising:
retrieving, by the computer-based system, a work injury form from a healthcare database;
creating, by the computer-based system, a real-time communication link between a first communication device operated by an employee and a second communication device operated by a first healthcare provider;
providing, by the computer-based system, an employee graphical user interface comprising an employee input field and an employee audiovisual display field, wherein the first communication device displays the employee graphical user interface;
providing, by the computer-based system, a healthcare provider graphical user interface comprising a healthcare provider input field and a healthcare provider audiovisual display field, wherein the second communication device displays the healthcare provider graphical user interface;
receiving, by the computer-based system, medical information of the employee from at least one of the first communication device or the second communication device;
populating, by the computer-based system, the work injury form with the medical information; and
providing, by the computer-based system, the work injury form to at least one of the first healthcare provider, an employer, or a regulatory body.

2. The article of manufacture of claim 1, further comprising:
authorizing, by the computer-based system, the employee to access the computer-based system.

3. The article of manufacture of claim 2, further comprising:
selecting, by the computer-based system, the first healthcare provider from a group of available healthcare providers; and
contacting, by the computer-based system, the second communication device operated by the first healthcare provider.

4. The article of manufacture of claim 3, wherein the medical information comprises at least one of medical history information and injury information.

5. The article of manufacture of claim 3, wherein the first healthcare provider is not authorized to prescribe medication.

6. The article of manufacture of claim 1, further comprising:
receiving, by the computer-based system, medical treatment instructions from the first healthcare provider; and
providing, by the computer-based system, the medical treatment instructions to the employee.

7. The article of manufacture of claim 1, further comprising:
selecting, by the computer-based system, a second healthcare provider from a group of available healthcare providers, wherein the second healthcare provider is authorized to prescribe medications;
contacting, by the computer-based system, a third communication device operated by the second healthcare provider; and
creating, by the computer-based system, a real-time communication link between the first communication device and the third communication device.

8. A computer-based telemedicine system for work-related injuries comprising:
a processor,
a tangible, non-transitory memory configured to communicate with the processor, the tangible, non-transitory memory having instructions stored thereon that, in response to execution by the processor, cause the processor to perform operations comprising:
retrieving, by the processor, a work injury form from a healthcare database;
creating, by the processor, a real-time communication link between a first communication device operated by an employee and a second communication device operated by a first healthcare provider;
providing, by the computer-based system, an employee graphical user interface comprising an employee input field and an employee audiovisual display field, wherein the first communication device displays the employee graphical user interface;
providing, by the computer-based system, a healthcare provider graphical user interface comprising a healthcare provider input field and a healthcare provider audiovisual display field, wherein the second communication device displays the healthcare provider graphical user interface;
receiving, by the processor, medical information of the employee from at least one of the first communication device or the second communication device;
populating, by the processor, the work injury form with the medical information; and
providing, by the processor, the work injury form to at least one of the first healthcare provider, an employer, or a regulatory body.

9. The computer-based telemedicine system of claim 8, further comprising:
authorizing, by the processor, the employee to access the computer-based telemedicine system.

10. The computer-based telemedicine system of claim 9, further comprising:
selecting, by the processor, the first healthcare provider from a group of available healthcare providers; and
contacting, by the processor, the second communication device operated by the first healthcare provider.

11. The computer-based telemedicine system of claim 10, wherein the medical information comprises at least one of medical history information and injury information.

12. The computer-based telemedicine system of claim 10, wherein the first healthcare provider is not authorized to prescribe medications.

13. The computer-based telemedicine system of claim 8, further comprising:
receiving, by the processor, medical treatment instructions from the first healthcare provider; and
providing, by the processor, the medical treatment instructions to the employee.

14. The computer-based telemedicine system of claim 8, further comprising:
selecting, by the processor, a second healthcare provider from a group of available healthcare providers, wherein the second healthcare provider is authorized to prescribe medications;
contacting, by the processor, a third communication device operated by the second healthcare provider; and
creating, by the processor, a real-time communication link between the first communication device and the third communication device.

15. A method comprising:
retrieving, by a computer-based system, a work injury form from a healthcare database;
selecting, by the computer-based system, a first healthcare provider from a group of available healthcare providers;
contacting, by the computer-based system, a first communication device operated by a first healthcare provider;
creating, by the computer-based system, a real-time communication link between the first communication device and a second communication device operated by an employee;
providing, by the computer-based system, an employee graphical user interface comprising an employee input field and an employee audiovisual display field, wherein the first communication device displays the employee graphical user interface;
providing, by the computer-based system, a healthcare provider graphical user interface comprising a healthcare provider input field and a healthcare provider audiovisual display field, wherein the second communication device displays the healthcare provider graphical user interface;
receiving, by the computer-based system, medical information of the employee from at least one of the first communication device or the second communication device;
populating, by the computer-based system, the work injury form with the medical information; and
providing, by the computer-based system, the work injury form to at least one of the first healthcare provider, an employer, or a regulatory body.

16. The method of claim 15, further comprising:
authorizing, by the computer-based system, the employee to access the computer-based system.

17. The method of claim 16, wherein the medical information comprises at least one of medical history information and injury information.

18. The method of claim 16, wherein the first healthcare provider is not authorized to prescribe medications.

19. The method of claim 15, further comprising:
receiving, by the computer-based system, medical treatment instructions from the first healthcare provider; and
providing, by the computer-based system, the medical treatment instructions to the employee.

20. The method of claim 15, further comprising:
selecting, by the computer-based system, a second healthcare provider from a group of available healthcare providers, wherein the second healthcare provider is authorized to prescribe medications;
contacting, by the computer-based system, a third communication device operated by the second healthcare provider; and
creating, by the computer-based system, a real-time communication link between the first communication device and the third communication device.

21. An article of manufacture, comprising:
a non-transitory, tangible computer-readable storage medium having instructions stored thereon that, in response to execution by a computer-based system, cause the computer-based system to perform operations comprising:
retrieving, by the computer-based system, a work injury form from a healthcare database;
creating, by the computer-based system, a real-time communication link between a first communication device and a second communication device;
providing, by the computer-based system, an graphical user interface, wherein at least one of the first communication device or the second communication device displays the graphical user interface;
receiving, by the computer-based system, medical information from at least one of the first communication device and the second communication device;
populating, by the computer-based system, the work injury form with the medical information; and
providing, by the computer-based system, the populated work injury form to at least one of the healthcare provider, an employer, or a regulatory body.

22. The article of manufacture of claim 21, wherein the work injury form is selected in response to at least one of an employee's identity, an employer's identity, or the employee's location.

23. The article of manufacture of claim 21, wherein the first communication device is operated by an employee and the second communication device is operated by a healthcare provider, and wherein the healthcare provider is not authorized to prescribe medications.

24. A computer-based telemedicine system for work-related injuries comprising:
a processor; and
the article of manufacture of claim 21.
